(19) United States
(12) Patent Application Publication
(10) Pub. No.: US 2018/0021231 A1
(43) Pub. Date: Jan. 25, 2018

(54) METHOD OF CONTROLLING SWEAT PRODUCTION

(71) Applicant: Conopco, Inc., d/b/a UNILEVER, Englewood Cliffs, NJ (US)

(72) Inventors: Douglas BOVELL, Bishopton (GB); Richard Livesey EVANS, Liverpool (GB); Clive Roderick HARDING, Little Neston (GB); Bijan HARICHIAN, Brookfield, CT (US); Jennifer ROBERTSON, Stirlingshire (GB); Ian Peter STOTT, Wirral (GB)

(73) Assignee: Conopco, Inc., d/b/a UNILEVER, Englewood Cliffs, NJ (US)

(21) Appl. No.: 15/521,014

(22) PCT Filed: Oct. 22, 2015
(86) PCT No.: PCT/EP2015/074488

§ 371 Date: Apr. 21, 2017

(30) Foreign Application Priority Data
Oct. 30, 2014 (EP) 14191106.5

(51) Int. Cl. A61K 8/35; A61K 8/34; A61K 8/73; A61Q 15/00

(52) U.S. Cl. CPC A61K 8/35; A61Q 15/00; A61K 8/345; A61K 8/34; A61K 8/731; A61K 2800/30; A61K 2800/87

(57) ABSTRACT

A cosmetic method of reducing perspiration comprising the topical application of a composition comprising a non-pore blocking antiperspirant agent of general formula (I): Ar—CHₐCHₐ—X wherein Ar is an aryl group, a is 1 or 2, and X is a sub-structure of formula CH(OH)—Y or CO.Z, wherein Y is a C₁-C₁₀ hydroxyalkyl group and Z is a C₁-C₁₀ hydroxyalkyl group or a C₁-C₁₀ oxyalkyl group.

The present invention is in the field of antiperspirancy, in particular methods of achieving antiperspirancy without gland blockage. The invention is of particular value in treating heavy sweating and/or hyperhydrosis.

There have been several publications published relating to the reduction of sweating using actives that do not block the sweat glands or pores.

WO 02/011690 (Unilever) discloses an antiperspirancy method whereby calcium channels with the secretory coil cells of the eccrine glands are blocked, thereby controlling sweat production at its source.

U.S. Pat. No. 8,618,160 B2 (Rose U) discloses wipes containing glycopyrrolate, a muscarinic anticholinergic, as a treatment for hyperhidrosis.

Certain anticholinergic drugs have also been used to treat hyperhidrosis, with varying degrees of success. These drugs include Ditropan® (oxybutynin), Probanthine® (propantheline bromide) and Cogentin® (benzotropine).

US 2008/0207737 (Zinger) discloses a topical composition for antiperspirant benefit comprising varying levels of oxybutynin.

In a first aspect of the invention, there is provided a cosmetic method of reducing perspiration comprising the topical application of a composition comprising a non-pore blocking antiperspirant agent of general formula I: Ar—CHₐCHₐ—X, wherein Ar is an aryl group, a is 1 or 2, and X is a sub-structure of formula CH(OH)—Y or CO.Z, wherein Y is a C₁-C₁₀ hydroxyalkyl group and Z is a C₁-C₁₀ hydroxyalkyl group or a C₁-C₁₀ oxyalkyl group.

In a second aspect of the invention, there is provided a non-pore blocking antiperspirant agent as defined in the first aspect of the invention for use as a treatment for perspiration, in particular excessive perspiration.

In a third aspect of the invention, there is provided an antiperspirant composition comprising a non-pore blocking antiperspirant agent selected from 6-gingerol, 6-gingerdiol or C₁-C₄ alkyl cinnamate and a carrier fluid comprising a C₂-C₆ vicinal diol.

In a fourth aspect of the invention, there is provided an antiperspirant composition as described in the third aspect of the invention and a dispenser for said composition, the dispenser comprising containment means and application means for the composition.

In the cosmetic method described in the first aspect of the invention, the composition preferably comprises a carrier fluid and more preferably a carrier fluid comprising a C₂-C₆ vicinal diol.

The aforementioned cosmetic method is a non-therapeutic method. In cosmetic methods, the non-pore blocking antiperspirant agent is typically applied as part of a cosmetic composition comprising a cosmetically acceptable carrier fluid.

In the second aspect of the invention, the non-pore blocking antiperspirant agent as defined in the first aspect of the invention may be used for the cosmetic or therapeutic treatment of perspiration, in particular excessive perspiration. Preferably, the non-pore blocking antiperspirant agent is used in conjunction with a carrier fluid and more preferably with a carrier fluid comprising a C₂-C₆ vicinal diol.

In the third aspect of the invention, preferred compositions comprise a non-pore blocking antiperspirant agent selected from 6-gingerol or 6-gingerdiol and a carrier fluid comprising a C₂-C₆ vicinal diol.

Herein, excessive perspiration should be understood to refer to that condition otherwise known as hyperhidrosis.

Herein, all percentages, parts, and ratios are by weight, unless otherwise indicated.

Herein, references to an amount of a compound or an active refer to the total amount of compounds or actives of the type indicated.

Herein, “application” and “applied” relate to application to the surface of the human body, in particular the underarm regions, unless the context dictates otherwise.

The invention typically involves the topical application of the non-pore blocking antiperspirant agent to the underarm regions of the human body, otherwise known as the axillae. From there, it is believed that the antiperspirant agent permeates to the secretory coil of the sweat glands, said permeation generally being enhanced by other components with which the antiperspirant agent is formulated. Without wishing to be bound by theory, it is believed that the antiperspirant agent reduces sweat production at its source, the sweat glands themselves.

The non-pore blocking antiperspirant agent used in accordance with the present invention is of general formula I (Ar—CHₐCHₐ—X) as previously defined.

Herein, an “aryl” group is an optionally substituted phenyl group. Preferably, para to the X group and at solely one position meta to the X group, the aryl group is non-substituted or has a substituent selected from OH or OCH₃ and at all other positions the aryl group is non-substituted.

For the avoidance of doubt, a non-substituted position on an aryl group in Formula I merely bears an H atom.

For the avoidance of doubt, when “a” in Formula I equals 1, a double bond exists between the indicated carbon atoms and when “b” in Formula I equals 2, a single bond exists between the indicated carbon atoms.

When X in general formula I is a sub-structure of formula CH(OH)—Y, Y is preferably of formula CH₂CH(OH)R, wherein R is a C₁-C₆ alkyl group (preferably a C₅ linear, unbranched alkyl group).

When X in general formula I is a sub-structure of formula CO.Z, Z is preferably of formula CH₂CH(OH)R, wherein R is a C₁-C₆ alkyl group (preferably a C₅ linear, unbranched alkyl group) or Z is a C₁-C₄ oxyalkyl group.

Herein, an “oxyalkyl group” should be understood to be an O atom followed by a non-substituted alkyl group, typically linear and unbranched.

In preferred embodiments, the non-pore blocking antiperspirant agent is selected from 6-gingerol, 6-gingerdiol, or a C₁-C₄ alkyl cinnamate (preferably ethyl cinnamate).

In particularly preferred embodiments, the non-pore blocking antiperspirant agent is selected from 6-gingerol or 6-gingerdiol.

6-gingerol has the systematic name (5S)-5-hydroxy-1-(4-hydroxy-3-methoxyphenyl)-3-decanone.

6-gingerdiol has the systematic name (5S)-1-(4-hydroxy-3-methoxyphenyl)-decane-3,5-diol.

The non-pore blocking antiperspirant agent is preferably used from a composition comprising it at a level of from 0.1 to 20%, preferably from 0.2 to 10% and more preferably from 0.5 to 5%.

Compositions used in accordance with the present invention may optionally include pore-blocking antiperspirant agents, including conventional astringent aluminium-containing antiperspirant salts.

Such conventional astringent aluminium-containing antiperspirant salts include aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates. Especially preferred aluminium halohydrate salts, known as activated aluminium chlorohydrates, are described in EP 6,739 (Unilever PLC and NV). Zirconium aluminium chlorohydrate actives are also preferred materials, as are the so-called ZAG (zirconium-aluminium-glycine) complexes, for example those disclosed in U.S. Pat. No. 3,792,068 (Procter and Gamble Co.). Zinc phenol sulphonate may also be used, preferably at up to 3% by weight of the composition.

When employed, preferred levels of incorporation of conventional astringent aluminium-containing antiperspirant salts are from 0.5% to 60%, particularly from 5% to 40%, and especially from 5% or 10% to 30% or 35% of the composition.

In certain preferred embodiments of the invention, conventional astringent aluminium-containing antiperspirant salts are not employed.

The carrier fluid used in conjunction with the present invention should be cosmetically acceptable and aid in the delivery of the non-pore blocking antiperspirant agent to the surface of the human body. The non-pore blocking antiperspirant agent is typically suspended or dissolved in the carrier fluid.

The carrier fluid may comprise from 10 to 99% of the composition.

In preferred aspects of the present invention, the carrier fluid for the non-pore blocking antiperspirant agent comprises a C₂-C₆ vicinal diol.

Herein, the C₂-C₆ vicinal diol is a compound with from 2 to 6 carbon atoms in which two neighbouring carbon atoms bear hydroxyl groups.

Examples of C₂-C₆ vicinal diols include ethylene glycol, propylene glycol, glycerol and hexane-1,2-diol.

Preferably, the C₂-C₆ vicinal diol excludes compounds comprising more than 3 hydroxyl groups.

Preferably the C₂-C₆ vicinal diol is a C₂-C₄ vicinal diol, i.e. a compound with from 2 to 4 carbon atoms in which two neighbouring carbon atoms bear hydroxyl groups.

Preferably, the C₂-C₆ or C₂-C₄vicinal diol excludes compounds comprising more than 3 hydroxyl groups.

Preferably, the C₂-C₆ vicinal diol excludes mono-saccharides and reduced mono-saccharides.

The content of C₂-C₆ vicinal diol in compositions suitable for use in accordance with the invention is preferably from 1 to 50%, more preferably from 2 to 40% and most preferably from 5 to 25%.

A preferred additional component of the carrier fluid is ethanol. This may comprise up to 80% of the total composition, but is typically restricted to less than 70%, and often less than 60%. When employed ethanol typically comprises at least 10%, preferably at least 20%, and more preferably at least 40% by weight of total composition. Each of these preferred minimum levels of ethanol may be limited by the aforementioned maximum levels of ethanol.

A preferred additional component of the carrier fluid is water, especially when employed in conjunction with ethanol, to give an aqueous ethanol carrier fluid. Water may comprise up to 90% of the total composition, but is typically restricted to less than 60%, and often less than 50%. When employed water typically comprises at least 10%, preferably at least 20%, and more preferably at least 30% by weight of total composition. Each of these preferred minimum levels of water may be limited by the aforementioned maximum levels of water.

A preferred additional component is a fragrance, typically at a level of from 0.1 to 5% of the total composition. In compositions also comprising water, the fragrance is preferably accompanied by a fragrance solubiliser, typically a non-ionic surfactant used a concentration of from 0.1 to 5% of the total composition.

A further optional component is an organic anti-microbial agent. Such agents may be selected from any of those known in the art, provided reasonable skill is used to avoid any incompatibilities. An organic anti-microbial agent is a particularly preferred additional component when the composition used does not include a conventional, astringent aluminium containing antiperspirant salt. When employed, organic anti-microbial agents are typically used at a level of from 0.1 to 5% by weight of the composition.

Thickening agents may be employed in compositions of the invention. Such agents increase the viscosity of or solidify the carrier fluid in which the antiperspirant agent is typically suspended or dissolved.

Thickening agents may be selected from any of those known in the art, provided reasonable skill is used to avoid any incompatibilities. A preferred class of thickeners, especially for compositions also comprising water, are hydroxyalkyl celluloses, such as hydroxypropyl cellulose. When employed, thickening agents are typically used at a level of from 0.1 to 40%. When a hydroxyalkyl cellulose thickening agent is employed, this is typically used at a level of from 0.2 to 10% by weight.

Dispensers suitable for use with the present invention comprise containment means and application means for the composition. They also typically comprise means for getting the composition from its containment means to its application means.

The containment means is preferably a reservoir for the composition and the application means is preferably a surface from which the composition may be massaged into the skin.

Preferred dispensers are able to function without the consumer needing to touch the composition with his or her hands in order for it to be applied, this feature enhancing dosing accuracy.

### EXAMPLES

The compositions detailed in Table 1 may be prepared by methods known in the art. The pH of the resulting composition is adjusted to 6 to 7 with 0.2N sodium hydroxide solution. The level of hydroxypropyl cellulose may be adjusted to gain the desired level of viscosity for the composition.

TABLE 1

| | Example | | | | |
|---|---|---|---|---|---|
| | 1 | 2 | 3 | 4 | 5 |
| 6-gingerol | 1.0 | 0.5 | — | — | — |
| 6-gingerdiol | — | — | 1.0 | 0.5 | — |
| Ethyl | — | — | — | — | 1.0 |
| cinnamate | | | | | |
| Hydroxypropyl | 1.7 | 1.7 | 1.5 | 1.0 | 1.0 |
| cellulose | | | | | |
| Propylene | 10.0 | 10.0 | 12.0 | 8.0 | 8.0 |
| glycol | | | | | |
| Glycerol | 1.0 | 1.0 | 0.8 | 1.2 | 1.2 |
| Ethanol | 50 | 50 | 60 | 40 | 40 |
| Water | To 100 | To 100 | To 100 | To 100 | To 100 |

1. A cosmetic method of reducing perspiration comprising the topical application of a composition comprising a non-pore blocking antiperspirant agent of general formula I:
Ar—CHₐCHₐ—X
wherein Ar is an aryl group, a is 1 or 2, and X is, a sub-structure of formula CH(OH)—Y or CO.Z, wherein 1 is a C₁-C₁₀ hydroxyalkyl group and Z is a C₁-C₁₀ hydroxyalkyl group or a C₁-C₁₀ oxyalkyl group.

2. A cosmetic method according, to claim 1, wherein the non-pore blocking antiperspirant agent of general formula I has an aryl group such that pare to the X group and at solely one position meta to the X group, the aryl group is non-substituted or has a substituent selected from OH or OCH₃ and at all other, positions the aryl group is non-substituted.

3. A cosmetic method according to claim 1, wherein X in general formula I is as a sub-structure of formula CH(OH)—Y′ or CO.Z, wherein Y′ is of formula CH₂CH(OH)R, wherein R is a C₁-C₆ alkyl group (preferably a C₅ linear, unbranched alkyl group), and Z is as defined in claim 1.

4. A cosmetic method according to any claim 1, wherein X in general formula I is a sub-structure of formula CH(OH)—Y or CO.Z′, wherein Y is as defined in claim 1 and Z′ is of formula CH₂CH(OH)R, wherein R is a C₁-C₆ alkyl group (preferably a C₅ linear unbranched alkyl group) or is a C₁-C₄ oxyalkyl group.

5. A cosmetic method according to claim 1, wherein the non-pore blocking antiperspirant agent is selected from 6-gingerol, 6-gingerdiol, or ethyl cinnamate.

6. A cosmetic method according to claim 5, wherein the non-pore blocking antiperspirant agent is selected from 6-gingerol or 6-gingerdiol.

7. A cosmetic method according to claim 1, wherein a conventional astringent aluminium-containing antiperspirant salt is not also applied.

8. A non-pore blocking antiperspirant agent as described in claim 1 for use as a treatment for perspiration in particular excessive perspiration.

9. An antiperspirant composition comprising a non-pore blocking antiperspirant agent selected from 6-gingerol, 6-gingerdiol or C₁-C₄ alkyl cinnamate and a carrier fluid comprising a C₂-C₆ vicinal diol.

10. A composition according to claim 9, wherein the non-pore blocking antiperspirant agent comprises from 0.2 to 10% of the composition, the carrier fluid comprises from 10 to 99% of the composition, and the C₂-C₆ vicinal dial from 2 to 40% of the composition, all percentages being by weight.

11. A composition according to claim 9, comprising a C₂-C₄ vicinal diol.

12. A composition according to claim 9, wherein the carrier fluid comprises water and ethanol.

13. A composition according to claim 9, comprising a thickening agent, preferably a hydroxyalkyl cellulose thickening agent.

14. A composition according to claim 9, wherein the non-pore blocking antiperspirant agent selected from 6-gingerol or 6-gingerdiol.

15. A product comprising a composition according to claim 9 and dispenser for said composition, the dispenser comprising containment means and application means for the composition.

16. A product according to claim 15, wherein the dispenser is able to function without the consumer needing to touch the composition with his or her hands in order for it to be applied.
